(19) United States
(12) Patent Application Publication
(10) Pub. No.: US 2006/0235241 A1
(43) Pub. Date: Oct. 19, 2006

(54) Process for the carbonylation of a conuugated diene

(76) Inventors: Eit Drent, Amsterdam (NL); Rene Ernst, Amsterdam (NL); Willem Wabe Jager, Amsterdam (NL)

Correspondence Address: SHELL OIL COMPANY, P O BOX 2463, HOUSTON, TX, 772522463, US

(21) Appl. No.: 10/557,309

(22) PCT Filed: May 13, 2004
(86) PCT No.: PCT/EP04/50794

§ 371 Date: Nov. 18, 2005

(30) Foreign Application Priority Data
May 22, 2003 (EP) 03076567.1
Feb. 26, 2004 (EP) 04251065.1

(51) Int. Cl. C07C 67/36; C07C 45/49

(52) U.S. Cl. 560/207; 568/454

(57) ABSTRACT

A process for the carbonylation of a conjugated diene, which involves reacting the conjugated diene with carbon monoxide and a co-reactant having a mobile hydrogen atom in the presence of a catalyst system including: (a) a source of palladium; and (b) a bidentate diphosphine ligand of formula II,
R¹R²>P¹—R³ₘ—R—R⁴ₙ—P²<R⁵R⁶ (II)
wherein P¹ and P² represent phosphorus atoms;
R¹, R², R⁵ and R⁶ independently represent the same or different optionally substituted organic radical containing a tertiary carbon atom through which each radical is linked to the phosphorus atom;
R³ and R⁴ independently represent the same or different optionally substituted methylene groups;
R represents an organic group having the bivalent bridging group C¹—C² through
which R is connected to R³ and R⁴;
m and n independently represent a natural number in the range of from 0 to 4, wherein the rotation about the bond between the carbon atoms C¹ and C² of the bridging group is restricted a temperature in the range of from 0° C. to 250° C., and wherein the dihedral angle between the plane occupied by the three atom sequence composed of C¹, C² and the atom directly bonded to C¹ in the direction of P¹, and the plane occupied by the three atom sequence C¹, C² and the atom directly bonded to C² in the direction of P², is in the range of from 0 to 120°; and (c) a source of an anion.

The present invention relates to a process for the carbonylation of a conjugated diene. Carbonylation reactions of conjugated dienes are well known in the art. In this specification, the term carbonylation refers to a reaction of a conjugated diene under catalysis by a transition metal complex in the presence of carbon monoxide and a co-reactant. In this process, the carbon monoxide as well as the co-reactant add to the diene, as for instance described in WO-A-03/031457.

Under the conditions usually employed for the carbonylation, conjugated dienes may also form dimers and/or telomers, as for instance described in WO-A-03/040065. This side reaction is highly undesired, as it reduces the yield of the desired carbonylation products. The selectivity towards carbonylation products over telomerisation products is further referred to herein as chemoselectivity.

Other than the need to achieve an as high as possible chemoselectivity, there is also the desire to achieve a particularly high selectivity towards one of several possible isomeric carbonylation products, further referred to herein as regioselectivity. For the carbonylation of conjugated dienes, the regioselectivity towards a linear product, i.e. towards reaction at the primary carbon atom, is often desired, as the branched products usually have no industrial use, whereas the linear products are important intermediates, for instance in the synthesis of adipic acid derivatives for use in polyamides.

WO-A-03/031457 discloses a process for the carbonylation of conjugated dienes, whereby the conjugated diene is reacted with carbon monoxide and a compound having a mobile hydrogen atom, for instance hydrogen, water, alcohols and amines in the presence of a catalyst system based on (a) a source of palladium cations, (b) a phosphorus-containing ligand of the formula (I)
Q¹>P—(CH₂)ₙ—PQ²Q³ (I)
wherein Q¹ is a bivalent radical which together with the phosphorus atom to which it is linked represents an unsubstituted or substituted 2-phospha-adamantane group or derivative thereof, wherein one or several of the carbon atoms are replaced by heteroatoms, Q² and Q³ independently represent a monovalent radical having 1-20 atoms or jointly bivalent radical having 2-20 atoms, and n is 4 or 5, and mixtures thereof.

Although exhibiting a high overall activity, the catalysts described in WO-A-03/031457 only provide a limited chemoselectivity and low yield. The disclosed carbonylation reaction yields a mixture of the several possible isomeric products, whereby the regioselectivity of the reaction is not disclosed in WO-A-03/031457. Furthermore, the described process requires the use of a large amount of palladium and ligand to achieve at least satisfactory turnover numbers, which makes the process costly to operate. Further, the product mixtures obtained need to undergo substantive purification and/or separation from byproducts and ligand remainders, which is undesirable in an industrial process.

Accordingly, there remains the need to provide for a catalyst system that combines a higher chemoselectivity and a higher regioselectivity for the linear carbonylation products, while also giving a high turn over and yield employing a lower amount of palladium to increase the overall efficiency of the process. Such a combination would also avoid having to subject the product mixture to a substantive purification to remove telomeric and polymeric by-products as well as the non-linear products.

It has now been found that the above identified process for the carbonylation of a conjugated diene with a coreactant having at least one mobile hydrogen atom can be very effectively performed in the presence of a different catalytic system as set out below.

### SUMMARY OF THE INVENTION

Accordingly, the subject invention provides a process for the carbonylation of a conjugated diene, comprising reacting the conjugated diene with carbon monoxide and a co-reactant having a mobile hydrogen atom in the presence of a catalyst system including:

(a) a source of palladium; and

(b) a bidentate diphosphine ligand of formula II,
R¹R²>P¹—R³ₘ—R—R⁴ₙ—P²<R⁵R⁶ (II)

wherein P¹ and P² represent phosphorus atoms; R¹, R², R⁵ and R⁶ independently represent the same or different optionally substituted organic group containing a, tertiary carbon atom through which each group is linked to the phosphorus atom;

R³ and R⁴ independently represent the same or different optionally substituted methylene groups;

R represents an organic group comprising the bivalent bridging group C¹—C² through which R is connected to R³ and R⁴;

m and n independently represent a natural number in the range of from 0 to 3,

wherein the rotation about the bond between the carbon atoms of the bridging group C¹ and C² of the bridging group is restricted at a temperature in the range of from 0° C. to 250° C., and wherein the dihedral angle between the plane occupied by the three atom sequence composed of C¹, C² and the atom directly bonded to C¹ in the direction of P¹, and the plane occupied by the three atom sequence C¹, C² and the atom directly bonded to C² in the direction of P², is in the range of from 0 to 120°; and

(c) a source of an anion.

In the process according to the invention, suitable sources for palladium of component (a) include palladium metal and complexes and compounds thereof such as palladium salts, for example the salts of palladium and halide acids, nitric acid, sulphuric acid or sulphonic acids; palladium complexes, e.g. with carbon monoxide or acetylacetonate, or palladium combined with a solid material such as an ion exchanger. Preferably, a salt of palladium and a carboxylic acid is used, suitably a carboxylic acid with up to 12 carbon atoms, such as salts of acetic acid, propionic acid and butanoic acid, or salts of substituted carboxylic acids such as trichloroacetic acid and trifluoroacetic acid. A very suitable source is palladium (II) acetate, or palladium (II) salts of the acids corresponding to the carbonylation product of the diene substrates, such as for instance palladium (II) pentenoate in the case of 1,3-butadiene as substrate.

The bidentate diphosphine ligand (b) has a structure according to formula (II) whereby the rotation about the bond between C¹ and C² is restricted at the temperature range of the reaction, and wherein the dihedral angle between the plane occupied by the three atom sequence composed of the atom bonded to C², C¹ and the atom directly bonded to C¹ in direction of P¹, and the plane occupied by the by the three atom sequence C¹, C² and the atom directly bonded to C² in direction of P² is in the range of from 0 to 120°.

The terms bond and rotation are as defined in Hendrickson, Cram and Hammond, Organic Chemistry, 3^{rd} Edition, 1970, pages 175 to 201. Rotation according to the subject invention means that the atoms attached to C¹ and C² respectively rotate about the axis that runs through the centre of the bond between C¹ and C².

The rotation about a bond is called “free” when the rotational barrier is so low that different conformations are not perceptible as different chemical species on the time scale of the experiment. The inhibition of rotation of groups about a bond due to the presence of a sufficiently large rotational barrier to make the phenomenon observable on the time scale of the experiment is termed hindered rotation or restricted rotation (as defined in IUPAC Compendium of Chemical Terminology, 2^{nd} Edition (1997), 68, 2209).

A suitable experiment can for instance be an ¹H-NMR-experiment as described in Hendrickson, Cram and Hammond, Organic Chemistry, 3^{rd} Edition, 1970, pages 265 to 281 and in F. A. Bovey, Nuclear Magnetic Resonance Spectroscopy, (New York, Academic Press, 1969), p. 1-20, provided that there are hydrogen atoms present in the ligand that will exhibit a suitable shift influenced by the bond between C¹ and C².

According to the subject invention, there is no free rotation about the bond between C¹ and C² at the temperature range at which the subject process is conducted. This temperature range may conveniently be in between 0° C. to 250° C., but preferably the subject process is conducted in the range of from 10° C. to 200° C., and yet more preferably in the range of from 15° C. to 150° C., and again more preferably in the range of from 18° C. to 130° C.

Accordingly, the rotation about the bond C¹—C² of the bidentate ligand is hindered or restricted at the temperature range of the subject process. Suitably the rotation is determined at ambient temperature.

The bridging group R comprises a chain of 2 optionally substituted carbon atoms C¹ and C². These carbon atoms C¹ and C² form the direct bridge between R¹R²P¹—R³ₘ— and —R⁴ₙ—P²R⁵R⁶, so that the phosphorus atoms P¹ and P² and the optionally substituted methylene groups R³ and R⁴ are connected via the bridging group C¹—C² to form the diphosphine ligands (b).

Although many different restricted conformations are possible for the subject ligands, a particular dihedral angle was found to be of high importance for the activity of the catalyst system. A dihedral angle is generally defined as the angle formed by two intersecting planes. The dihedral angle according to the subject process is the angle formed by the plane occupied by the three atom sequence composed of the three atoms C², C¹ and the atom directly bonded to C¹ in direction of P¹, and the plane occupied by the three atom sequence C¹, C² and the atom directly bonded to C² in direction of P² is in the range of from 0 to 120°, of the four atom sequence (atom directly bonded to C¹ in direction of P¹)—C¹—C²-(atom directly bonded to C² in the direction of P²). “In the direction of P¹ or P²” herein has the meaning that the relevant atom is situated in that part of the ligand chain that connects C¹ and P¹, or C² and P², respectively.

For instance, in the case that m and n are equal to 1, the dihedral angle is the angle between the plane occupied by the three atom sequence R³—C¹—C² of the four atom sequence R³—C¹—C²—R⁴ and the other three atoms C¹—C²—R⁴ of the four atom sequence R³—C¹—C²—R⁴. Each plane is understood to run through the central points of the respective atoms.

In the case that m and n of formula (II) should equal 0, the four atom sequence would accordingly be P¹—C¹—C²—P², and the two planes would be defined as P¹—C¹—C² and C¹—C²—P².

In the ligands according to the subject process, the dihedral angle as defined above is ranging from 0° to 120°. Since a higher catalytic activity of the catalyst system is thereby obtainable, the dihedral angle preferably is in the range of from 0° to 70, yet more preferably in the range of from 0° to 15°, and most preferably in the range of from 0° to 5°.

Without wishing to be bound to any particular theory, it is believed that ligands allowing rotation about the bond C¹-C² are less able to form a conformationally stable bidentate complex with the palladium centre. As a result, the bidentate complex might compete with a monodentate complex, thereby reducing the steric strain on the metal complex and hence reducing the catalytic activity of the complex.

The difficulty to obtain a stable bidentate complex is also illustrated by the increased amounts of ligands required in order to obtain a suitably high amount of the catalytically active chelate complex, and by the higher instability of the ligands under reaction conditions.

The bond formed between C¹ and C² may be a saturated or an unsaturated bond as occurring in ethylenically unsaturated or aromatic compounds. In the case of a saturated bond connecting C¹ and C², R can be expressed by C¹R′R″—C²R′″R″″, and the bidentate diphosphine ligand according to the present invention is thus suitably characterised by formula III
R¹R²P¹—R³ₘ—C¹R′R″—C²R′″R″″—R⁴ₙ—P²R⁵R⁶ (III).
In this embodiment, R′ and R″, and R″′ and R″″ represent hydrogen or the same or different optionally substituted organic group, provided that only one of R′ and R″, and only one of R′″ and R″″ is hydrogen. If C¹ and C² are connected by an ethylenically unsaturated double bond, C¹ and C² also cannot rotate freely. In this case, R can be expressed by C¹R′═C²R″, and the bidentate diphosphine ligand according to the present invention is thus suitably characterised by formula IV
R¹R²P¹—R³ₘ—C¹R′═C²R″—R⁴ₙ—P²R⁵R⁶ (IV).
If the bond between C¹ and C² is an ethylenically unsaturated bond, the ligand chain connecting P¹ and P² via C¹ and C² may in principally exist in two isomeric forms, a trans-configuration, and a cis-configuration. According to the above definition, in the trans-configuration the dihedral angle is about 180°, whereas in the cis-configuration, the dihedral angle is about 0°.

The substituents R′ to R″″ in formula III or IV can themselves be independent substituents, thus only connected to each other via the carbon atoms C¹ and C², or preferably have at least one further connection. The substituents may further comprise carbon atoms and/or heteroatoms.

The restriction of the free rotation may conveniently be achieved by the bridging group C¹-C² forming part of a molecular structure that impedes rotation about the bond C¹-C² at ambient temperature, and more preferably at a temperature range from 0 to 250° C., and preferably from 15 to 150° C. This molecular structure may conveniently be for instance a) an ethylenically unsaturated double bond, wherein the rotation is impeded by the energetically advantageous overlap of n-bonds, and/or b) a cyclic hydrocarbyl structure, in which the rotation is restricted due to the steric interaction of substituents R′ to R″″, or due to steric strain induced by a cyclic structure formed by R′ to R″″ together, or by combination of the above factors, such as in aromatic or non-aromatic cyclic structures. Conformational stability and hence rigidity may also c) be achieved if the nature of the substituents R′ and R″, and/or R″′ and R″″ is such that even if not connected to each other they impede rotation about the bond C¹—C², for instance by strong steric interactions. To this goal, preferably, none of R′ to R″″ in formula III or IV represent hydrogen.

R preferably is a cyclic hydrocarbyl structure that is optionally substituted by hetereoatoms, yet more preferably an aliphatic or aromatic hydrocarbyl structure. This structure may be part of an optionally further substituted saturated or unsaturated polycyclic structure, which also optionally may contain heteroatoms such as nitrogen, sulphur, silicon or oxygen atoms.

Suitable structures R include for instance substituted cyclohexane, cyclohexene, cyclohexadiene, substituted cyclopentane, cyclopentene or cyclopentadiene, all of which may optionally contain heteroatoms such as nitrogen, sulphur, silicon or oxygen atoms, with the proviso that the rotation about the bond C¹-C² is restricted, that the dihedral angle is in the range of from 0° to 120°, and that there is no rotation about the bond formed by C¹ and C² induced by conformational changes, as for instance in highly restrained acetal structures such as 2,2-dimethyl-1,3-dioxolane.

In one particularly preferred embodiment, R represents a divalent polycyclic hydrocarbyl ring structure. Such polycyclic groups are particularly preferred due to the high conformational stability and hence high restriction against free rotation about the bond between C¹ and C². Examples of such particularly preferred hydrocarbyl groups include norbornyl, norbornadienyl, isonobornyl, dicylcopentadienyl, octahydro-4,7-methano-1H-indenemethanyl, α- and β-pinyl, and 1,8-cineolyl, all of which may optionally be substituted, or contain heteroatoms as defined above.

In case that the bidentate ligand may have chiral centers, it may be in any R,R-, S,S- or R,S-meso form, or mixtures thereof. Both meso forms and racemic mixtures can be employed, provided that the dihedral angle is in the range of from 0 to 120°.

In the diphosphine of formula II, R preferably represents an optionally substituted divalent aromatic group which is linked to the phosphorus atoms via the groups R³ and R⁴.

Such an aromatic cyclic structure is preferred due to its rigidity, and to a dihedral angle being generally in the range of 0 to 5°.

The aromatic group can be a monocyclic group, such as for example a phenyl group or a polycyclic group, such as for example a naphthyl, anthryl or indyl group. Preferably, the aromatic group R contains only carbon atoms, but R can also represent an aromatic group wherein a carbon chain is interrupted by one or more hetero atoms, such as nitrogen, sulphur or oxygen atom in for example a pyridine, pyrrole, furan, thiophene, oxazole or thiazole group. Most preferably the aromatic group R represents a phenyl group or naphtylene group.

Optionally the aromatic group is substituted. Suitable substituents include groups containing hetero-atoms such as halides, sulphur, phosphorus, oxygen and nitrogen. Examples of such groups include chloride, bromide, iodide and groups of the general formula —O—H, —O—X, —CO—X, —CO—O—X, —S—H, —S—X, —CO—S—X, —NH₂, —NHX, —NO₂, —CN, —CO—NH₂, —CO—NHX, —CO—NX₂ and —Cl₃, in which X independently represents alkyl groups having from 1 to 4 carbon atoms like methyl, ethyl, propyl, isopropyl and n-butyl.

When the aromatic group is substituted it is preferably substituted with one or more aryl, alkyl or cycloalkyl groups, preferably having from 1 to 10 carbon atoms. Suitable groups include methyl, ethyl, trimethyl, iso-propyl, tetramethyl and iso-butyl, phenyl and cyclohexyl.

Most preferably, however, the aromatic group is non-substituted and only linked to the groups R³ and R⁴ which connect it with the phosphorus atoms. Preferably the alkylene groups are connected at adjacent positions, for example the 1 and 2 positions, of the aromatic group.

The symbols m and n in formula II, III and IV independently may represent a natural number in the range of from 0 to 3. If the m and n are 0, then the phosphorus atoms P¹ and P² are directly connected to bridge formed by the carbon atoms C¹ and C². If one of m or n equals 0, then either C¹ or C² will be directly connected to P¹ or P². Without wishing to be bound to any particular theory, it is believed that the effect resulting from the particular arrangement of the central bridge formed by C¹ and C² on the phosphorus atoms, and hence on the catalyst complex, will be diluted by the presence of a larger number of groups R³ and/or R⁴. Also, it is believed that if both m and n equal 0, the distance between the phosphorus atoms may be rather short, such that the ligand binds less strongly to the palladium centre atom of the catalyst complex.

Accordingly, due to generally good catalyst activity found with such ligands, m preferably equals 0 or 1, whereas n preferably is in the range of from 1 to 3, more preferably from 1 to 2 and most preferably 1.

If m and/or n have a value above 1, then several optionally substituted groups R³ and R⁴ connect P¹ and P² to R. These different may then be the same or individually different groups. Hence, R³ and/or R⁴ preferably are lower alkylene groups (by lower alkylene groups is understood alkylene groups comprising from 1 to 4 carbon atoms). These alkylene groups can be substituted, for example with alkyl groups or heteroatoms, or non-substituted, and may for instance represent methylene, ethylene, trimethylene, iso-propylene, tetramethylene, iso-butylene and tert-butylene, or may represent methoxy, ethoxy and similar groups. Most preferably, at least one of R³ and/or R⁴ is a methylene group.

Particularly suitable aromatic groups include aryl groups such as disubstituted phenyl or naphthyl groups, and substituted alkyl phenyl groups such as tolyl and xylyl groups. Preferred due to the easy synthetic availability and good solvability of the formed catalyst complex in the reaction medium are tolyl and xylyl groups, wherein the methylene substituent or methylene substituents at the aromatic ring serve as groups R³ and/or R⁴. Most preferably, C¹ and C² are part of an aromatic ring, whereas at least one of R³ and/or R⁴ represent methylene groups attached to the ring atoms C¹ and C².

Accordingly, an especially preferred ligand family according to the subject invention is that wherein C¹ and C² are part of a phenyl ring; m is 0 or 1; n is 1, and R³ and R⁴ are methylene groups. In yet another especially preferred ligand family due to easy synthetic accessibility, m and n equal 1. Accordingly, such ligands based on the 1,2-di(phosphinomethyl)benzene or 1-P-phosphino-2-(phosphinomethyl)-benzene groups are particularly suited for the subject process due to the high rigidity of the aromatic backbone, easy synthetic availability, and due to the very good results obtained with the derived catalyst system.

Other than the structure of the backbone, the direct ligand environment of the phosphorus atoms has also been found to have a strong effect in the selectivity and activity of the subject process. In the ligands that are used for the subject process, R¹, R², R⁵ and R⁶ independently may represent the same or a different optionally substituted organic group containing a tertiary carbon atom through which each group is linked to the phosphorus atom.

For the purposes of the subject invention, the term “organic group” represents an unsubstituted or substituted, aliphatic, aromatic or araliphatic radical having from 1 to 30 carbon atoms, which is connected to the phosphorus atom by a tertiary carbon atom, i.e. a carbon atom being bonded to the phosphorus and to three substituents other than hydrogen.

The organic groups R¹, R², R⁵ and R⁶ may each independently be a monovalent group, or R¹ and R² together and/or R⁵ and R⁶ together may be divalent groups. The groups may further contain one or more heteroatoms such as oxygen, nitrogen, sulfur or phosphorus and/or be substituted by one or more functional groups comprising for example oxygen, nitrogen, sulfur and/or halogen, for example by fluorine, chlorine, bromine, iodine and/or a cyano group.

The organic groups R¹, R², R⁵ and R⁶ may only be connected to each other via the phosphorus atom, and preferably have from 4 to 20 carbon atoms, and yet more preferably from 4 to 8 carbon atoms.

The tertiary carbon atom through which each of the groups is connected to the phosphorus atom can be substituted with aliphatic, cycloaliphatic, or aromatic substituents, or can form part of a substituted saturated or non-saturated aliphatic ring structure, all of which may contain heteroatoms, such as for instance I 1-adamantyl groups or derivatives thereof wherein carbon atoms in the structure have been replaced by oxygen atoms. Preferably the tertiary carbon atom is substituted with alkyl groups, thereby making the tertiary carbon atom part of a tertiary alkyl group, or by ether groups. Examples of suitable organic groups are tert-butyl, 2-(2-methyl)butyl, 2-(2-ethyl)butyl, 2-(2-phenyl)butyl, 2-(2-methyl)pentyl, 2-(2-ethyl)pentyl, 2-(2-methyl-4-phenyl)pentyl, 1-(1-methyl)cyclohexyl and 1-adamantyl groups.

Although the groups R¹, R², R⁵ and R⁶ may be each individually different organic groups, due to the use of lower amounts of different raw materials in the synthesis the groups R¹, R², R⁵ and R⁶ preferably represent the same tertiary organic group. Yet more preferably, the groups R¹, R², R⁵ and R⁶ represent tert-butyl groups or 1-adamantyl groups, the most preferred being tert-butyl groups. Accordingly, the subject invention pertains to the process, wherein R¹, R², R⁵ and R⁶ each represents a tertiary butyl group. Especially preferred bidentate diphosphine are thus 1,2-bis(ditert-butylphosphinomethyl)benzene (also describes as bis[di(tert-butyl)phosphino]-o-xylene or dtbx ligand) and 2,3-bis(ditert-butylphosphinomethyl)naphtene.

Although very good results have been obtained using ligands wherein groups R¹, R², R⁵ and R⁶ represent the same tertiary alkyl groups such as tert-butyl groups, these ligands can however be difficult to obtain on an industrial scale due to the required use of metal organic compounds such as Grignard reactants.

Similarly good results were obtained with diphosphine ligands, wherein R¹ and R² together and/or R⁵ and R⁶ represent a divalent group that is directly attached to the phosphorus atom via two tertiary carbon atoms. This divalent group may have a monocyclic or a polycyclic structure. Diphosphines containing phosphorous atoms bearing such divalent groups have the advantage that they are accessible via a different synthetic route involving reacting phosphines at milder conditions, which makes them more accessible on an industrial scale. Accordingly, R¹ and R² together and/or R⁵ and R⁶ together may also represent an optionally substituted divalent cycloaliphatic group, wherein the cycloaliphatic group is linked to the phosphorus atom via two tertiary carbon atoms. R¹ together with R², and/or R⁵ together with R⁶ are in each case preferably a branched cyclic, hetero-atom unsubstituted or substituted divalent alkyl group having from 4 to 10 atoms in the alkylene chain, in which the CH₂- groups may also be replaced by hetero groups, for example —CO—, —O—, —SiR₂— or —NR— and in which one or more of the hydrogen atoms may be replaced by substituents, for example aryl groups.

Examples of preferred divalent groups are unsubstituted or substituted C₄—C₃₀-alkylene groups in which CH₂- groups may be replaced by hetero groups such as —O—, include include 1,1,4,4-tetramethyl-buta-1,4-diyl-, 1,4-dimethyl-1,4-dimethoxy-buta-1,4-diyl-, 1,1,5,5-tetramethyl-penta-1,5-diyl-, 1,5-dimethyl-1,5-dimethoxy-penta-1,5-diyl-, 3-oxa-1,5-dimethoxy-penta-1,5-diyl-, 3-oxa-1,1,5,5-tetramethyl-penta-1,5-diyl-, 3-oxa-1,5-dimethyl-1,5-dimethoxy-penta-1,5-diyl- and similar divalent radicals.

Particularly suitable monocyclic structures including R¹ and R² together, and/or R⁵ and R⁶ together are for instance optionally heteroatom-substituted 2,2,6,6-tetrasubstituted phosphinan-4-one or -4-thione structures. Ligands comprising such structures may be conveniently obtained under mild conditions Ligands comprising such structures may be conveniently obtained under mild conditions as described in Welcher and Day, Journal of Organic Chemistry, J. Am. Chem. Soc., 27 (1962) 1824-1827.

For instance, a bidentate diphosphine with identical organic groups R¹, R², R⁵ and R⁶ may conveniently be obtained by reacting the compound
H₂P—(R³)ₘ—C¹R′R″—C²R′″R″″—(R⁴)ₙPH₂
with a compound
(Z¹Z²C)═(CZ³)—(C═Y)—(CZ⁴)═(CZ⁵Z⁶),
whereby Z¹, Z², Z⁵ and Z⁶ represent optionally heteroatom-substituted organic groups, Z³ and Z⁴ represent optionally heteroatom-substituted organic groups or hydrogen, and whereby Y represents oxygen or sulfur. An example for such a compound is 2,6-dimethyl-2,5-heptadien-4-one (also known as diisopropylidene acetone, or phorone). If more than a single compound is employed, ligands with different groups comprising R¹ and R², and comprising R⁵ and R⁶ are formed.

A suitable polycyclic structure including R¹ and R², and/or R⁵ and R⁶ is for instance the 2-phosphatricyclo[3.3.1.1{3,7}]decyl group that is substituted in 1,3 and 5 position (thus providing the tertiary carbon atoms through which the group is connected to the phosphorous atom), or a derivative thereof in which one or more of the carbon atoms are replaced by heteroatoms. Tricyclo[3.3.1.1{3,7}]decane is the systematic name for a compound more generally known as adamantane. The 1,3,5-trisubstituted 2-phospha-tricyclo[3.3.1.1{3,7}decyl group or a derivative thereof will thus be referred to as “2-PA” group (as in 2-phosphadamantyl group) throughout the specification.

The 2-PA group is substituted on one or more of the 1, 3, 5 positions, and optionally also on the 7 position, with a monovalent organic group R⁷ from 1 to 20 atoms, preferably from 1 to 10 carbon atoms, yet more preferably from 1 to 6 carbon atoms. Examples of R⁷ include methyl, ethyl, propyl and phenyl.

More preferably, the 2-PA group is substituted on each of the 1, 3, 5 and 7 positions, suitably with identical groups R⁷, yet more preferably with methyl groups. The 2-PA group further contains preferably additional heteroatoms other than the 2-phosphorus atom in its skeleton. Suitable heteroatoms are oxygen and sulphur atoms. More suitably, these heteroatoms are found in the 6, 9 and 10 positions. The most preferred bivalent radical is thus the 2-phospha-1,3,5,7-tetramethyl-6,9,10-trioxadamantyl group.

The bidentate ligands used in the process according to the invention can be prepared as described for example in WO 01/68583, or in Chem. Commun. 2001, pages 1476 to 1477 (Robert I. Pugh et. Al.). Accordingly, the subject invention also pertains to a process, wherein R¹ and R² together and/or R⁵ and R⁶ together in formula (II) are part of an optionally heteroatom substituted 1,3,5-trisubsituted 2-phospha-adamantane structure, or part of an optionally heteroatom substituted 2,2,6,6-tetrasubstituted-phosphinan-4-one, or part of an optionally heteroatom substituted 2,2,6,6-tetra-substituted-phosphinan-4-thione.

The bidentate ligands can be prepared in the meso- and rac-form, all of which are suitable.

Especially preferred diphosphine ligands according to the subject invention are compounds according to formula (II), wherein R¹ together with R², and R⁵ together with R⁶, together with the respective phosphorus atoms P¹ or P² form 2-phospha-1,3,5,7-tetramethyl-6,9,10-trioxadamantyl groups, or a 2,2,6,6-tetramethyl phosphinan-4-one, and wherein the backbone structure R³—C¹—C²—R⁴ is a α-phosphinotoluyl, 1,2-xylyl or 2,3-naphtyl structure, i.e. wherein R³, R⁴ are methylene groups, m is 1 and n 0 or 1, and the bond C¹—C² is part of a phenyl ring, due to the very good results obtained with these ligands; the most preferred ligand of this embodiment being that wherein n and m equal 1.

Bidentate diphosphine ligands that can conveniently be used in the subject process have for instance been disclosed in WO-A-96/19434, WO-A-98/42717, WO-A-01/68583 and WO-A-01/72697 and include the highly preferred ligands 1,2-P,P′-di(2-phospha-1,3,5,7-tetramethyl-6,9,10-trioxatricyclo[3.3.1.1{3.7}decyl)-methylene-benzene (also sometimes referred to as 1,2-P,P′-di(2-phospha-1,3,5,7-tetramethyl-6,9,10-trioxatricyclo[3.3.1.1{3,7}decyl)-o-xylene) and 1,2-P,P′-di-(2-phospha-1,3,5,7-tetra(ethyl)-6,9,10-trioxatricyclo[3.3.1.1{3.7}decyl)-methylene-benzene.

In WO-A-01/68583, there is disclosed a process for the carbonylation of ethylenically unsaturated compounds having 3 or more carbon atoms by reaction with carbon monoxide and an hydroxyl group containing compound, in the presence of a catalyst system including:

(a) a source of palladium;

(b) a bidentate diphosphine as applied in the present process, and,

(c) a source of anions derived from an acid having a PKₐ of less than 3, as measured at 18° C. in an aqueous solution; the process being carried out in the presence of an aprotic solvent. The preferred hydroxyl containing compounds according to WO-A-01/68583 are water and alkanols. Notably, the carbonylation of conjugated dienes not mentioned in this document.

Without wishing to be bound to any particular theory, it is believed that ethylenically unsaturated compounds not being conjugated dienes, and conjugated dienes react in carbonylation reactions via completely different intermediate complexes with the catalyst metal centre. The conjugated dienes according to the subject process are believed to form an intermediate n-allyl-complex with the metal centre of the catalyst complex, which intermediate complex may react further. Ethylenically unsaturated compounds, which are not conjugated dienes however cannot form such a n-allyl-complex.

Hence, a skilled reader would not be able to transfer the results found for the carbonylation of ethylenically unsaturated compounds without conjugated double bonds to the carbonylation of conjugated dienes, in particular with respect to reactivity, chemoselectivity and/or regioselectivity of the formed products.

Contrary to the above-identified ligands, the ligands disclosed in WO-A-03/31457 do not have a restricted rotation about the bond connecting the phosphorus atoms according to the subject invention. Due to the C₄- and C₅-alkylene backbone of these ligands, they should show a free rotation already at room temperature about the dihedral axis, as the presence of hydrogen substituents at the bridging atoms is considered to not result in a large energetic difference between the different possible conformations to prevent the ligands from rotation under the conditions usually employed for carbonylation reactions.

The ratio of moles of bidentate diphosphine, i.e. catalyst component (b), per mole atom of palladium cations, i.e. catalyst component (a), ranges from 0.5 to 10, preferably from 0.8 to 8, and yet more preferably from 1 to 5.

Very good results have also been obtained with bidentate diphosphine ligands whereby R¹ and R² are each individually organic groups only connected to each other via a phosphorus atom, whereas R⁵ and R⁶ together represent a bivalent organic group that is bonded to the second phosphorus atom via two tertiary carbon atoms. Such ligands with unsymmetrical substitution at the two phosphorus atoms have not been described before, nor their use in catalyst compositions useful for carbonylation reactions. Accordingly, the subject invention also pertains to a bidentate diphosphine ligand of formula II,
R¹R²P¹—(R³)ₘ—R—(R⁴)ₙ—P²R⁵R⁶ (II)
wherein P¹ and P² represent phosphorus atoms;
R¹ and R² independently represent the same or different optionally substituted organic radical containing a tertiary carbon atom through which each radical is linked to the phosphorus atom, and which radicals are solely connected to each other via the phosphorus atom P¹;
R⁵ and R⁶ together represent an organic bivalent radical linked to the phosphorus atom P² via tertiary carbon atoms;
R³, and R⁴ independently represent the same or different optionally substituted organic group;
and m and n independently represent a natural number in the range of from 0 to 3. Preferably, R³ and R⁴ are substituted methylene groups.

The subject invention further provides for catalyst compositions comprising: (a) a source of a metal of group VIII, and (b) the novel bidentate diphosphine ligand formula II, wherein P¹ and P² represent phosphorus atoms;

R¹ and R² independently represent the same or different optionally substituted organic radical containing a tertiary carbon atom through which each radical is linked to the phosphorus atom, and which radicals are solely connected to each other via the phosphorus atom P¹; R⁵ and R⁶ together represent an organic bivalent radical linked to the phosphorus atom P² via tertiary carbon atoms; R³, and R⁴ independently represent hydrogen or the same or different optionally substituted organic group; and m and n independently represent a natural number in the range of from 0 to 3. Suitable group VIII metals include Pd, Pt and Rh, preferred being Pd and Pt, the most preferred being Pd for carbonylation of conjugated dienes.

The good results obtained with all ligands according to the subject invention proves the general inventive concept that a particularly high reactivity and selectivity can be obtained if R¹, R² and R⁵ and R⁶ are attached via tertiary carbon atoms to the respective phosphorus atoms.

Although these novel ligands might be useful in a number of processes, for instance in a catalyst composition for carbonylation reactions for ethylenically unsaturated compounds, or preferably for conjugated dienes, this use requires that the ligand should be in a cis-configuration, as set out above.

Accordingly, the subject invention also pertains to the use of the novel bidentate diphosphine ligand as set-out above in a catalyst system for the carbonylation of a conjugated diene, whereby in the ligand the rotation about the bond between C¹ and C² is restricted at ambient temperature, and wherein the dihedral angle between the plane occupied by the three atom sequence composed of the three atom sequence C², C¹ and the atom directly bonded to C¹ in direction of P¹, and the plane occupied by the three atom sequence C¹, C² and the atom directly bonded to C² in direction of P² is in the range of from 0 to 120°.

Such a ligand is for instance 1-P-(1,3,5,7-tetramethyl-1,3,5-trimethyl-6,9,10-trioxa-2-phosphatricyclo(3.3.1.1{3,7}]decyl-2-(di-tert-butylphosphinomethyl)benzene.

The ratio of moles of bidentate diphosphine, i.e. catalyst component (b), per mole atom of palladium, i.e. catalyst component (a), is not critical. Preferably it ranges from 0.1 to 100, more preferably from 0.5 to 10.

However, for a more preferred catalyst the active species is believed to be based on an equimolar amount of bidentate diphosphine ligand per mole palladium. Thus, the molar amount of bidentate diphosphine ligand per mole palladium is preferably in the range of 1 to 3, more preferably in the range of 1 to 2, and yet more preferably in the range of 1 to 1.5. In the presence of oxygen, slightly higher amounts may be beneficial.

The subject process permits to react conjugated dienes with carbon monoxide and a co-reactant. The conjugated diene reactant has at least 4 carbon atoms. Preferably the diene has from 4 to 20 and more preferably from 4 to 14 carbon atoms. However, in a different preferred embodiment, the process may also be applied to molecules that contain conjugated double bonds within their molecular structure, for instance within the chain of a polymer such as a synthetic rubber.

The conjugated diene can be substituted or non-substituted. Preferably the conjugated diene is a non-substituted diene. Examples of useful conjugated dienes are the 1,3-butadienes, conjugated pentadienes, conjugated hexadienes, cyclopentadiene and cyclohexadiene, all of which may be substituted. Of particular commercial interest are 1,3-butadiene and 2-methyl-1,3-butadiene (isoprene).

The feed containing the diene reactant does not necessarily have to be free of admixture with alkenes, since the carbonylation reaction of the present invention is particularly selective for diene feeds. Even an admixture with up to 30 mol %, preferably with up to 5 mol % of alkynes, basis the diene reactant, can be tolerated in the feed.

The ratio (v/v) of diene and co-reactant in the feed can vary between wide limits and suitably lies in the range of 1:0.1 to 1:500.

The co-reactant according to the present invention may be any compound having a mobile hydrogen atom, and capable of reacting as nucleophile with the diene under catalysis. The nature of the co-reactant largely determines the type of product formed. A suitable co-reactant is water, a carboxylic acid, alcohol, ammonia or an amine, a thiol, or a combination thereof. Inasmuch as the co-reactant is water, the product obtained will be an ethylenically unsaturated carboxylic acid. Ethylenically unsaturated anhydrides are obtained inasmuch as the co-reactant is a carboxylic acid. For an alcohol co-reactant, the product of the carbonylation is an ester. Similarly, the use of ammonia (NH₃) or a primary or secondary amine RNH₂ or R′R″NH will produce an amide, whereas the use of a thiol RSH will produce a thioester. In the above-defined co-reactants, R, R′ and/or R″ represent optionally heteroatom-substituted organic radicals, preferably alkyl, alkenyl or aryl radicals. When ammonia or amines are employed, a small portion of these co-reactants will react with acids present under formation of an amide and water. Hence, in the case of ammonia or amine-co-reactants, there is always water present.

Preferably the carboxylic acid co-reactant has the same number of carbon atoms as the diene reactant, plus one.

Preferred alcohol co-reactants are alkanols with 1 to 20, more preferably with 1 to 6 carbon atoms per molecule, and alkanediols with 2-20, more preferably 2 to 6 carbon atoms per molecule. The alkanols can be aliphatic, cycloaliphatic or aromatic. Suitable alkanols in the process of the invention include methanol, ethanol, ethanediol, n-propanol, 1,3-propanediol, iso-propanol, 1-butanol, 2-butanol (sec-butanol), 2-methyl-1-propanol (isobutanol), 2-methyl-2-propanol (tert-butanol), 1-pentanol, 2- pentanol, 3-pentanol, 2-methyl-1-butanol, 3-methyl-l-butanol (isoamyl alcohol), 2-methyl-2-butanol (tert-amyl alcohol), 1-hexanol, 2-hexanol, 4-methyl-2-pentanol, 3,3-dimethyl-2-butanol, 1-heptanol, 1-octanol, 1-nonanol, 1-decanol, 1,2-ethylene glycol and 1,3-propylene glycol, of which methanol is the most preferred due to the high turn over achievable and due to the particular usefulness of the obtained products.

Preferred amines have from 1 to 20, more preferably 1 to 6 carbon atoms per molecule, and diamines have from 2-20, more preferably 2 to 6 carbon atoms per molecule. The amines can be aliphatic, cycloaliphatic or aromatic. More preferred due to the high turnovers achieved are ammonia and primary amines. In the case that the anion (c) of the catalyst system is an acid, preferably the amount of ammonia or amine is less than stoichiometric based on the amine functionality. Inadvertently, when the coreactant is ammonia, and to a lesser extent a primary amine, a small amount of the acid present will react to an amide under liberation of water. Hence, there is also always a small amount of acid formed from the conjugated diene, carbon monoxide and the water, which in turn replaces acid converted to amide by the direct reaction as described above.

The thiol co-reactants can be aliphatic, cycloaliphatic or aromatic. Preferred thiol co-reactants are aliphatic thiols with 1 to 20, more preferably with 1 to 6 carbon atoms per molecule, and aliphatic dithiols with 2-20, more preferably 2 to 6 carbon atoms per molecule.

The source of anions (c) may be any source of anion suitable to catalyze the reaction. However, the source of anions preferably is an acid, more preferably a carboxylic acid, which can serve both as promoter component (c), as well as solvent for the reaction. Again more preferably, the source of anions is an acid having a pKₐ above 2.0 (measured in aqueous solution at 18° C.), and yet more preferably catalyst component (c) is an acid having a pKₐ above 3.0, and yet more preferably a pKₐ of above 3.6.

Examples of preferred acids include acetic acid, propionic acid, butyric acid, pentanoic acid, pentenoic acid and nonanoic acid, the latter three being highly preferred as their low polarity and high PKₐ was found to increase the reactivity of the catalyst system. Very conveniently the acid corresponding to the desired product of the reaction can be used as the catalyst component (c). Pentenoic acid is particularly preferred in case the conjugated diene is 1,3-butadiene. Catalyst component (c) can also be an ion exchanging resin containing carboxylic acid groups. This advantageously simplifies the purification of the product mixture.

The molar ratio of the source of anions, and palladium, i.e. catalyst components (c) and (b), is not critical. However, it suitably is between 2:1 and 10⁷:1 and more preferably between 10²:1 and 10⁶:1, yet more preferably between 10²:1 and 10⁵:1, and most preferably between 10²:1 and 10⁴:1 due to the enhanced activity of the catalyst system. Accordingly, if a co-reactant should react with the acid serving as source of anions, then the amount of the acid to co-reactant should be chosen such that a suitable amount of free acid is present. Generally, a large surplus of acid over the co-reactant is preferred due to the enhanced reaction rates.

The quantity in which the complete catalyst system is used is not critical and may vary within wide limits. Usually amounts in the range of 10⁻⁸ to 10⁻¹, preferably in the range of 10⁻⁷ to 10⁻² mole atom of palladium per mole of conjugated diene are used, preferably in the range of 10⁻⁵ to 10⁻² gram atom per mole. The process may optionally be carried out in the presence of a solvent, however preferably the acid serving as component (c) is used as solvent and as promoter.

The carbonylation reaction according to the present invention is carried out at moderate temperatures and pressures. Suitable reaction temperatures are in the range of 0-250° C., more preferably in the range of 50-200° C., yet more preferably in the range of from 80-150° C.

The reaction pressure is usually at least atmospheric. Suitable pressures are in the range of 0.1 to 15 MPa (1 to 150 bar), preferably in the range of 0.5 to 8.5 MPa (5 to 85 bar). Carbon monoxide partial pressures in the range of 0.1 to 8 MPa (1 to 80 bar) are preferred, the upper range of 4 to 8 MPa being more preferred. Higher pressures require special equipment provisions.

In the process according to the present invention, the carbon monoxide can be used in its pure form or diluted with an inert gas such as nitrogen, carbon dioxide or noble gases such as argon, or co-reactant gases such as ammonia.

Furthermore, the addition of limited amounts of hydrogen, such as 3 to 20 mol % of the amount of carbon monoxide used, promotes the carbonylation reaction. The use of higher amounts of hydrogen, however, tends to cause the undesirable hydrogenation of the diene reactant and/or of the unsaturated carboxylic acid product.

The subject process has the additional advantage, that with the exception of reactions wherein ammonia or amine co-reactants or halogen-containing co-reactants are employed, no nitrogen-containing compounds or halogen-containing compounds are required. As a result, the obtained products are substantially free from nitrogen-containing impurities or halogen-containing impurities.

Moreover, the dicarboxylic acid product composition only contains minor amounts of branched dicarboxylic acid product isomeres (such as α-methyl glutaric acid and/or α-ethyl succinic acid in the case of adipic acid product composition), and preferably less than 1.5 ppmw of nitrogen-containing impurities and less than 1.5 ppmw of halogen-containing impurities, yet more preferably less than 0.1 ppmw, and most preferably less than 1 ppbw of nitrogen-containing impurities and less than 1 ppbw of halogen-containing impurities. When 1,3-butadiene was converted, the adipic acid product composition could advantageously be employed in the synthesis of polyamide products, as it did contain less than 1.5 ppmw of each of glutaric acid and/or succinic acid, and as surprisingly the minor amounts of α-methyl glutaric acid and/or α-ethyl succinic acid present in the product composition did not cause significant problems in the manufacturing process, and may advantageously reduce the melt temperature of the polymer without negatively affecting other physical properties. Accordingly the adipic acid product contains preferably less than 0.1 ppmw of each of glutaric acid and/or succinic acid, more preferably less than 1 ppbw of each of glutaric acid and/or succinic acid. Therefore, the subject invention also preferably relates to the carbonylation product composition obtainable by the subject process, wherein the product composition contains α-methyl glutaric acid and/or α-ethylsuccinic acid, and less than 1.5 ppmw of nitrogen-containing impurities and less than 1.5 ppmw of halogen-containing impurities, and less than 1.5 ppmw of each of glutaric acid and/or succinic acide.

The invention will be illustrated by the following non-limiting examples.

### EXAMPLE 1

### Preparation of 1-P-(1,3,5,7-tetramethyl-6,9,10-trioxa-2-phospha Tricyclo[3.3.1.1^{{3,7}}]decyl)-2-[di-tert-butyl-phosphinomethyl)benzene Ligand

8.25 g (33 mmol) 2-bromobenzylbromide and 5 g (34.2 mmol) di-tert. butyl phosphine in 40 ml degassed acetonitrile were measured into a 100 ml glass reactor under an inert atmosphere, and then stirred for a period of 12 hours at ambient temperature. The acetonitrile was then removed in vacuo and 30 ml degassed toluene, 30 ml degassed water and 7.5 ml triethylamine were added. To this mixture 10 ml ethanol was added to improve phase separation. Upon phase separation, the upper layer containing the toluene was separated and evaporated to dryness. The remainder was 9 g (28.6 mmol, 87%) of (2-bromobenzyl)(di-tert-butyl)phosphine as a light yellow oil exhibiting a resonance peak in ³¹P NMR at +34.16 ppm.

2.5 g (7.9 mmol) of the thus obtained 2-bromobenzyl-(di-tert-butyl)phosphine, 2.24 g DABCO (20 mmol), 1.94 g 1,3,5-trimethyl-4,6,9-trioxa-2-phosphatricyclo-[3.3.1.1^{{3,7}}]decane (9 mmol) and 0.23 g Pd(PPh₃)₄ (0.2 mmol) in 10 ml toluene were added into a 250 ml glass vessel under inert atmosphere, and the content of the vessel was heated to 140° C. under stirring for 12 hours. The mixture was than allowed to cool to 100° C., and was then filtered. The filtrate was cooled to room temperature, then 30 ml of methanol added were added and the mixture cooled for a period for 12 hours to −35° C., 1-P-(1,3,5,7-tetramethyl-6,9,10-trioxa-2-phosphatricyclo[3.3.1.1^{{3,7}}]decyl)-2-(di-tertbutylphosphinomethyl)-benzene was isolated as yellow crystals (2.2 g, 4.9 mmol, 62%), and could be characterized by showing two distinct resonance signals in ³¹P NMR at +38.08 and −38.96 ppm. The ligand will be further referred to herein as α-dtb-2-pa-tolyl ligand, and represents a ligand according to formula II, wherein R=aryl, m=0, n=1, and the dihedral angle is about 0°.

### EXAMPLES 2-18 AND COMPARATIVE EXAMPLES A-D

### Batch Reactions for Carbonylation of Butadiene With Water

A 250 ml magnetically stirred autoclave, made of HASTELLOY C (HASTELLOY C is a trademark), was successively charged with acid in an amount as indicated in Table I below, 5 ml water, 0.1 mmol palladium acetate and the respective ligand in an amount as indicated in Table I below (in mmol).

In Examples 2-13 and Example 18, the ligand was 1,2-Bis(di-tert-butylphosphinomethyl)benzene (further referred to as dtbx, according to formula (II), R equals benzene, m=n=1, the dihedral angle is about 0°); in Example 14, the ligand was 2,3-bis(di-tert-butyl-phosphinomethyl)naphtalene (further referred to as dtbn, according to formula (II), R equals naphtalene, m=n=1, the dihedral angle is about 0°); in Example 15, the ligand was 1-P-(1,3,5,7-tetramethyl-6,9,10-trioxa-2-phospha-tricyclo[3.3.1.1{3,7}]decyl)-2-[di-tertbutylphosphinomethyl)benzene (α-pa-2-dtb-tolyl ligand as obtained in Example 1; according to formula (II), R equals benzene, m=0, n=1, the dihedral angle is about 0°); in Examples 16 and 17, the ligand was 1,2-Bis(P,P′-(1,3,5,7-tetramethyl-6,9,10-(2-phosphatrioxatricyclo[3.3.1.1{3.7}]decyl)methyl benzene (further referred to as 1,2-bpa-o-xylyl ligand; according to formula (II), R equals benzene, m=n=1, the dihedral angle is about 0°). In Example 18, the substrate was 2-methyl-butadiene (isoprene) instead of butadiene.

In Comparative Example A the ligand was 3-(di-tert-butylphosphino)-2-(di-tert-butylphosphinomethyl)-1-propene (not according to the subject invention; the rotation about the bonds C¹ and/or C² is not restricted); in Comparative Example B the ligand was 1,2-Bis-(9-phosphabicyclo[3.3.1]nonyl)ethane (not according to the subject invention; the rotation about the bonds C¹ and/or C² is not restricted, and the phosphorus atoms are not bearing tertiary substituents); in Comparative Example C 5 the ligand was 1,3-Bis(di-tert-butylphosphino)propanone (not according to the subject invention; the rotation about the bonds C¹ and/or C² is not restricted); in Comparative Example D the ligand was 1,2-Bis(dicyclohexylphosphinomethyl)benzene (not according to the subject invention; the phosphorus atoms are not bearing tertiary substituents).

The autoclave was then closed and evacuated and 20 ml butadiene was pumped in. The autoclave was pressurized with H₂ and/or CO and to partial pressures as indicated in Table I, sealed, heated to 135° C. and maintained at that temperature for 10 hours. Finally the autoclave was cooled and the reaction mixture was analysed with GLC.

It was found that in Examples 2-18 practically 100% of the initial substrate (butadiene) was converted to (pentenoic) acid within the 10-hour reaction time (in Example 18, isoprene was converted to methyl pentenoic acid), while in Comparative Examples A-D the conversion did not reach a level above 15%.

The initial carbonylation rate (mol per mol Pd per hour) of this batch operation, as presented in Table I, is defined for Examples 2-18 as the mean rate of carbon monoxide consumption (pressure drop) over the first 30% substrate consumption. For Comparative Examples A-D, which did not reach 40% substrate consumption, the initial carbonylation rate is defined as the mean rate of CO consumption over the first two hours.

TABLE I

| | | | | | | Initial |
|---|---|---|---|---|---|---|
| | | | | H₂ partial | CO partial | carbonylation |
| Exam- | | | | pressure | pressure | rate |
| ple | Ligand | Substrate | Solvent | MPa | MPa | mol/mol Pd/hr |
| 2 | dtbx [0.25] | butadiene | diglyme/3-pentenoic | — | 4.0 | 400 |
| | | | acid [25/25 ml] | | | |
| 3 | dtbx [0.25] | butadiene | hexanoic acid [40 ml] | — | 4.0 | 460 |
| 4 | dtbx [0.25] | butadiene | nonanoic acid [40 ml] | — | 4.0 | 700 |
| 5 | dtbx [0.25] | butadiene | propionic acid [40 ml] | — | 4.0 | 400 |
| 6 | dtbx [0.25] | butadiene | 3-pentenoic acid [40 ml] | — | 4.0 | 280 |
| 7 | dtbx [0.25] | butadiene | hexanoic acid [40 ml] | 1.0 | 4.0 | 740 |
| 8 | dtbx [0.25] | butadiene | hexanoic acid [40 ml] | 0.5 | 4.0 | 780 |
| | | | | | | Initial |
| Exam- | | | | H₂ partial | CO partial | carbonylation |
| ple | Ligand | Substrate | Solvent | pressure | pressure | rate |
| 9 | dtbx [0.25] | butadiene | hexanoic acid [40 ml] | — | 4.0 | 460 |
| 10 | dtbx [0.25] | butadiene | hexanoic acid [40 ml] | 0.2 | 4.0 | 560 |
| 11 | dtbx [0.25] | butadiene | hexanoic acid [40 ml] | — | 6.0 | 880 |
| 12 | dtbx [0.25] | butadiene | hexanoic acid [40 ml] | 0.5 | 6.0 | 1020 |
| 13 | dtbx [0.2] | butadiene | 3-pentenoic acid [35 ml] | — | 6.0 | 240 |
| 14 | dbtn [0.2] | butadiene | 3-pentenoic acid [35 ml] | — | 6.0 | 400 |
| 15 | α-pa-2-dtb-tolyl [0.5] | butadiene | nonanoic acid [35 ml] | — | 6.5 | 350 |
| 16 | 1,2-bpa-o-xylyl[0.2] | butadiene | nonanoic acid [35 ml] | — | 6.0 | 225 |
| | | | | | | Initial |
| Exam- | | | | H₂ | CO | carbon. |
| ple | Ligand | Substrate | Solvent | p. pres. | p. pres. | rate |
| 17 | 1,2-bpa-o-xylyl [0.5] | butadiene | 3-pentenoic acid [35 ml] | — | 6.0 | 450 |
| 18 | dtbx [0.25] | isoprene | hexanoic acid [40 ml] | — | 6.0 | 1200 |
| A | 3-(di-tert-butylphosphino)-2-(di- | butadiene | hexanoic acid [40 ml] | — | 4.0 | 30 |
| | tert-butylphosphino-methyl)-1- | | | | | |
| | propene [0.5 mmol] | | | | | |
| B | 1,2-Bis-(9-phosphabicyclo- | butadiene | hexanoic acid [40 ml] | — | 4.0 | 5 |
| | [3.3.1]nonyl)-ethane [0.5 mmol] | | | | | |
| C | 1,3-Bis(di-tert-butylphos- | butadiene | hexanoic acid [40 ml] | — | 4.0 | 45 |
| | phino)propanone [0.5 mmol] | | | | | |
| D | 1,2-Bis(dicyclohexylphosphino- | butadiene | hexanoic acid [40 ml] | — | 4.0 | 10 |
| | methyl)benzene [0.5 mmol] | | | | | |

### EXAMPLE 19 AND COMPARATIVE EXAMPLES E and F

### Batch Reactions for Carbonylation of Butadiene With Methanol to Pentenoate

A 250 ml magnetically stirred autoclave was successively charged with palladium acetate (0.1 mmol), 20 ml methanol, 40 ml pentenoic acid and 0.5 mmol ligand.

In Example 19 the same ligand was used as in Examples 1-13, and in Comparative Example E the same ligand was used as in Comparative Example B.

The autoclave was then closed and evacuated and flushed with nitrogen, and then 20 ml butadiene was pumped in. The autoclave was pressurized with CO to 6 MPa, sealed, heated to 135° C. and maintained at the temperature for 10 hours. In the comparative Examples E and F, no consumption of carbon monoxide was observed, and about 30% of the butadiene had reacted to a mixture of 4-vinylcyclohexene and butadiene polymer.

TABLE II
Reaction with methanol

| | | | | Initial | |
|---|---|---|---|---|---|
| | | | CO partial | carbonylation | |
| | | | pressure | rate | Conversion |
| Example | Ligand | Solvent | MPa | Mol/mol Pd/hr | Butadiene |
| 19 | dtbx [0.5 mmol] | 3-pentenoic acid [40 ml] | 6.0 | 1200 | 100% |
| E | 1,2-bis- (9-phosphabicyclo- | 3-pentenoic acid [40 ml] | 6.0 | — | 30%* |
| | [3.3.1]-nonylethane [0.5 mmol] | | | | |
| F | 1,4- (diphenylphosphino)- | 3-pentenoic acid [40 ml] | 6.0 | — | 30%* |
| | butane [0.5 mmol] | | | | |

*Butadiene was converted to a mixture of 4-vinylcyclohexene and butadiene polymer

### EXAMPLES 20-21 AND COMPARATIVE EXAMPLE G

### Batch Reactions for Carbonylation of Butadiene With Acid to Pentenoic Acid Via Anhydride

A 250 ml magnetically stirred autoclave was successively charged with 20 ml acetic acid, 40 ml diglyme, palladium acetate (0.25 mmol in Example 20 and 0.1 mmol in Example 21 and Comparative Example G), and 0.5 mmol of the respective ligand. In Examples 20 and 21, the same ligand was used as in Examples 1-13, and in Comparative Example G the same ligand was used as in Comparative Example A.

The autoclave was then closed and evacuated and 10 ml butadiene was pumped in.

The autoclave was pressurized with CO to 4 MPa, sealed, heated to 135° C. and maintained at that temperature for 10 hours. After cooling the contents was analysed with GLC.

The initial carbonylation rate was defined as for Examples 1-18 and Comparative Examples A-D.

In Example 20 the butadiene conversion to pentenoic acid was >90% while the acetic acid was converted to acetic anhydride for 35%. The initial carbonylation rate was 400 mol/mol Pd/hr.

In Example 21 the same conversions were measured as in Example 20 but the reaction rate was 900 mol/molPd/hr.

In Comparative Example G the butadiene conversion to pentenoic acid was 15% while the acetic acid was converted to acetic anhydride for 5%. The reaction rate was 60 mol/mol Pd/hr.

### EXAMPLE 22

### Semi Continuous Reaction for Producing Pentenoic Acid From Butadiene

A 1.2 l mechanically stirred autoclave was charged with 150 ml nonanoic acid and 5 ml water. The autoclave was degassed three times with CO at 3.0 MPa. Next the autoclave was pressurised with CO to 5.0 MPa, followed by adding 20 ml of butadiene. Next the catalyst, consisting of a solution of 0.1 mmol of palladium acetate and 0.5 mmol of 1,2-bis(di-tert-butylphosphinomethyl)benzene dissolved in 10 g nonanoic acid was injected. The injector was rinsed with a further 10 g of nonanoic acid.

Next butadiene and water at a rate of 40-50 mmol/h respectively, were continuously added to the reactor, which was heated to 130° C. over 30 minutes. When this temperature has been reached the pressure was adjusted to 8.0 MPa. These conditions were maintained for 68 hours. After cooling the mixture was distilled at 70-80° C. and 10 Pa, yielding 304 g of a mixture having the following composition as analysed with GLC.

TABLE III

| | |
|---|---|
| butenyl esters of pentenoic acid | 6.1 wt % |
| butenyl esters of nonanoic acid | 1.4 wt % |
| Cis/trans 3-pentenoic acid | 84.0 wt % |
| 2- and 4-pentenoic acid | 1.4 wt % |
| nonanoic acid | 6.9 wt % |

The carbonylation rate of this semi continuous operation is defined as mol of reacted butadiene per mol of Pd per hour, and the total turnover as mol of reacted butadiene per mol of Pd. Based on the above results the average carbonylation rate during the 68 hours of operation was 390 and the total turnover 26000.

### EXAMPLES 23-26

### Further Hydrocarboxylation of Batches of the Mixed Product of Example 20 to Adipic Acid

Four batches of 30 ml each of the mixed distilled product of Example 21 specified above were further reacted with CO and water as follows.

A 250 ml magnetically stirred autoclave, made of HASTELLOY C, was charged with water as specified in Table III below and with 30 ml of the distilled product of Example 21. Then 0.1 mol palladium acetate and 0.5 mol of the ligand 1,2-Bis(di-tert-butylphosphinomethyl)-benzene were added and the autoclave closed and evacuated. The autoclave was pressurized with H₂ and/or CO to partial pressures as indicated in Table III, sealed, heated to 135° C. and maintained at that temperature for 15 hours. Finally the autoclave was cooled and the reaction mixture was analysed with GLC.

The reaction mixture was almost completely composed of solid adipic acid. THF was added to form a slurry of adipic acid in THF. The THF phase was analysed by GLC and the conversion of pentenoic acid was determined from the residual pentenoic acid. In all experiments pentenoic acid conversion was higher than 90%. Selectivity to adipic acid was >95%.

The initial carbonylation rate (mol per mol of Pd per hour) of this batch operation, as presented in Table III, is defined as the mean rate of carbon monoxide consumption (pressure drop) over the first 30% substrate consumption.

TABLE IV

| | | | | | Initial |
|---|---|---|---|---|---|
| | | | H₂ | CO | carbonyla- |
| | | Induction | partial | partial | tion rate |
| | Water | time | pressure | pressure | mol/mol |
| Example | charge | (hr)** | MPa | MPa | Pd/hr |
| 23 | 5 ml | 6 | 10 | 40 | 610 |
| 24 | 5 ml | 5 | — | 60 | 700 |
| 25 | 7 ml | 10 | — | 65 | 730 |
| 26 | 2 + 5 ml* | <1 | — | 65 | 880 |

*5 ml were added after 1 hr reaction
**The induction time is caused by the butenyl pentenoic acid esters present in the feed (6.1 wt % according to Table II), which are initially converted to pentenoic acid and butadiene. At the low initial water concentration of Example 17 this conversion was rapidly achieved.

### EXAMPLES 27 AND 28

### Direct Carbonylation of Butadiene to Adipic Acid

In a first step a 250 ml magnetically stirred autoclave, made of HASTELLOY C, was successively charged with 35 ml pentenoic acid, 5 ml water, 0.1 mmol palladium acetate and 0.5 mmol of the ligand 1,2-Bis(di-tert-butylphosphinomethyl)benzene. The autoclave was then closed and evacuated and 20 ml butadiene was pumped in. The autoclave was pressurized to 6 MPa with CO, sealed, heated to 135° C. and maintained at that temperature for 10 hours. After cooling down the autoclave was opened and a sample taken, slurred with THF and analysed by GLC. It was found that practically 100% of the initial substrate (butadiene) was converted to (pentenoic) acid within the 10-hour reaction time.

In a second step, after cooling down, 7 ml of water was added to the autoclave and the autoclave was again pressurised with CO to 6 MPa, heated to 135° C. and maintained at that temperature for another 10 hours. After cooling, the contents were slurred in THF and analysed with GLC. It was found that the butadiene and the pentenoic acid were converted to adipic acid for more then 95%. The recovered yield by filtration was 69 grams.

The initial carbonylation rate (mol per mol of Pd per hour) of this batch operation, in both steps, is defined as the mean rate of carbon monoxide consumption (pressure drop) over the first 30% substrate consumption. The rate of the first step was 400 mol/mol Pd/hr. The rate of the second step was 550 mol/mol Pd/hr.

### EXAMPLE 29

### Direct Carbonylation of a Butane-butene-butadiene Feed Mixture to Adipic Acid

In a first step a 250 ml magnetically stirred autoclave, made of HASTELLOY C, was successively charged with a catalyst composition consisting of 35 ml of the product mixture of Example 21 (84 wt % of which was pentenoic acid), 5 ml water, 0.1 mmol palladium acetate and 0.5 mmol of the ligand 1,2-bis[di(tert-butyl)-phosphinomethyl]benzene. The autoclave was then closed and evacuated and 31 grams of a butane-butenes-butadiene feed mixture of the following composition was pumped in.

| Component | Mol % |
|---|---|
| Acetylene | 0.03 |
| Propane | 0.01 |
| Propene | 0.03 |
| Butane | 3.35 |
| Propyne/trans 2-butene | 6.54 |
| Cis-2 butene | 5.37 |
| 2-methyl propane | 0.91 |
| 1-butene | 8.72 |
| iso-butene | 28.13 |
| 1.3 butadiene | 45.44 |
| Pentane/1.2 butadiene | 0.44 |
| 2-methyl-2-butene | 0.77 |
| 3-methyl-1-butene | 0.05 |
| 2-methyl-1-butene | 0.17 |
| C₆₊ hydrocarbons | 0.03 |

The autoclave was pressurized to 6 Mpa with CO, sealed, heated to 135° C. and maintained at that temperature for 10 hours. After cooling down the autoclave was opened, a sample taken, slurred with THF and analysed by GLC. It was found that practically 100% of the initial substrate (butadiene) was converted to (pentenoic) acid within the 10-hour reaction time, while butene conversion did not reach 2%.

In a second step, 2 ml of water was added and the autoclave was closed again and evacuated to remove any remaining olefins originating from the BBB feed mixture, pressurised again with CO to 6 MPa and heated to 135° C. After 2.5 hours a further 5 ml of water was injected (using CO at 8 MPa) and the pressure and temperature maintained for another 8 hours. After cooling the solid contents were slurred out of the reactor with THF and re-crystallised to yield 43 grams of a solid, which when analysed by ¹H NMR (solvent d-DMSO) was shown to be >99% pure adipic acid. The initial carbonylation rate (mol per mol of Pd per hour) of this batch operation, in both steps, is defined as the mean rate of carbon monoxide consumption (pressure drop) over the first 30% substrate consumption.

The rate of the first step was 1150 mol/mol Pd/hr.

The rate of the second step was 200 mol/mol Pd/hr.

### EXAMPLE 30

### Semi Continuous Reaction for Producing Adipic Acid From Butadiene

A 1.2 1 mechanically stirred autoclave was charged with 150 ml nonanoic acid and 5 ml water. The autoclave was degassed three times with CO at 3.0 MPa. Next the autoclave was pressurised with CO to 5.0 MPa, followed by adding 20 ml of butadiene. Next the catalyst, consisting of a solution of 0.1 mmol of palladium acetate and 0.5 mmol of 1,2-bis(di-tert-butylphosphinomethyl)benzene dissolved in 10 g nonanoic acid was injected. The injector was rinsed with a further 10 g of nonanoic acid.

Next butadiene and water at a rate of 40-50 mmol/h respectively, were continuously added to the reactor, which was heated to 130° C. over 30 minutes. When this temperature has been reached the pressure was adjusted to 8.0 MPa. These conditions were maintained for about 10 hours, and samples taken at regular intervals. Once a TON of 30,000 mol pentenoic acid/mol catalyst, and a selectivity towards pentenoic acid of about 97% was achieved, the butadiene feed was stopped, and the remaining butadiene was allowed to react. Then water was added until the water concentration was about 10% w/w of the reactor mixture, and the reaction was continued under the same conditions as before(8.0 MPa CO pressure and 135° C.) until the pentenoic acid was fully converted.

After cooling and release of the pressure, the contents of the autoclave were slurred in THF and analysed with GLC. It was found that the pentenoic acid had been converted to adipic acid with a selectivity for more then 97%, and the overall selectivity starting from butadiene to adipic acid was 94%. The TON of the second reaction was 10,000 mol adipic acid/mol catalyst. The adipic acid prepared in this reaction contained less than 1.5 ppmw of nitrogen-containing impurities, and less than 1.5 ppmw of halogen-containing impurities, and less than 0.1 ppmw of glutaric acid and succinic acid.

### EXAMPLE 31

### Amidation of Butadiene

A HASTELLOY C (HASTELLOY is a registered trademark of Haynes International, Inc.) 250 ml autoclave was charged with 0.1 mmol palladium acetate and 0.5 mmol of the ligand 1,2-bis[di(tert-butyl)phosphinomethyl]benzene 0.1 mmol Pd(II) acetate and 34 ml pentenoic acid. The autoclave was then pressurized to 0.2 MPa (2 bar) with NH₃. Subsequently, 10 ml 1,3-butadiene were pumped into the reactor and then the reactor was pressurized to 6 MPa (60 bar) with carbon monoxide. Following sealing of the autoclave, its contents were heated to a temperature of 135° C. and maintained at that temperature for 7 hours. After cooling, a sample was taken from the contents of the autoclave and analysed by Gas Liquid Chromatography.

The 1,3-butadiene and the ammonia had been converted to 100%, with selectivity towards 2- and 3-penteneamide of about 99%, the remainder containing traces of pentenoic acid anhydride.

The above experiments show that the process for the carbonylation of conjugated dienes proceeds at high to very high turn over rates to complete conversion, and with high overall selectivity for the linear products, which incidentally also do not contain halogen-containing impurities, and with exception of the amidation products are also free from nitrogen-containing impurities.

Moreover, novel ligands and a process for their preparation are described, which provide alternative catalyst systems with ready accessibility.

1. A process for the carbonylation of a conjugated diene, which comprises reacting the conjugated diene with carbon monoxide and a co-reactant having a mobile hydrogen atom in the presence of a catalyst system including:
(a) a source of palladium; and
(b) a bidentate diphosphine ligand of formula II,
R¹R²>P¹—R³ₘ—R—R⁴ₙ—P²<R⁵R⁶ (II)
wherein P¹ and P² represent phosphorus atoms;
R¹, R², R⁵ and R⁶ independently, or R¹ and R² together and/or R⁵ and R⁶ together represent the same or different optionally substituted organic radical containing a tertiary carbon atom through which each radical is linked to the phosphorus atom;
R³ and R⁴ independently represent the same or different optionally substituted methylene groups;
R represents an organic group comprising the bivalent bridging group C¹-C² through which R is connected to R³ and R⁴;
m and n independently represent a natural number in the range of from 0 to 4,
wherein the rotation about the bond between the carbon atoms C¹ and C² of the bridging group is restricted at a temperature in the range of from 0° C. to 250° C., and wherein the dihedral angle between the plane occupied by the three atom sequence composed of C¹, C² and the atom directly bonded to C¹ in the direction of P¹, and the plane occupied by the three atom sequence C¹, C² and the atom directly bonded to C² in the direction of P², is in the range of from 0 to 120°; and
(c) a source of an anion.

2. The process of claim 1 wherein the source of anions (c) is an acid having a pKₐ of more than 3, measured at 18° C. in aqueous solution.

3. The process of claim 1, wherein the rotation is about the bond between the carbon atoms C¹ and C² is restricted at ambient temperature.

4. The process of claim 1, wherein R is an optionally substituted aromatic group.

5. The process of claim 1, wherein n is 1, and wherein m is 0 or 1.

6. The process of claim 1, wherein R³ and R⁴ represent methylene groups.

7. The process of claim 1, wherein an amount of 3 to 20 mol %, related to the carbon monoxide, of hydrogen is added.

8. The process of claim 1, wherein R¹, R², R⁵ and R⁶ each represent a tertiary butyl group.

9. The process of claim 1, wherein R¹ and R² together and/or R⁵ and R⁶ together are part of a 2-phospha-adamantane structure, a phosphinan-4-one structure, or a phosphinan-4-thione structure.

10. The process of claim 1, wherein the conjugated diene is 1,3-butadiene or 2-methyl-1,3-butadiene.

11. The process of claim 1, wherein the catalyst component (c) is present in a molar ratio to catalyst component (a) in the range of from 10²:1 to 10⁴:1.

12. The process of claim 1, wherein the diene is reacted with carbon monoxide and a co-reactant selected from the group of water and carboxylic acids in the presence of a catalyst system including:
(a) a source of palladium;
(b) a bidentate diphosphine of formula II,
R¹R²>P¹—R³—R—R⁴—P²<R⁵R⁶ (II),
wherein P represents a phosphorus atom; R¹, R², R⁵ and R⁶ independently represent the same or different optionally substituted organic groups containing a tertiary carbon atom through which the group is linked to the phosphorus atom; R³ and R⁴ independently represent optionally substituted alkylene groups and R represents an optionally substituted aromatic group;
(c) a source of anions derived from an acid having a pKₐ of more than 3, as measured at 18° C. in an aqueous solution.

13. The process of claim 1, wherein the reaction temperature is in the range of 50° C. to 250° C., the reaction pressure is in the range of 0.1 to 15 MPa, and the carbon monoxide partial pressure is in the range of 0.1 to 6.5 MPa.

14. A bidentate diphosphine ligand of formula II,
R¹R²>P¹—R³ₘ—R—R⁴ₙ—P²<R⁵R⁶ (II),
wherein P¹ and P² represent phosphorus atoms; R³ and R⁴ independently represent the same or different optionally substituted organic groups; R represents an organic group comprising the bivalent bridging group C¹-C² through which R is connected to R³ and R⁴, m and n independently represent a natural number in the range of from 0 to 4; wherein one of pairs R¹ and R² together, or R⁵ and R⁶ together independently represent the same or different optionally substituted organic radical containing a tertiary carbon atom through which each radical is linked to the phosphorus atom, and which radicals are solely connected to each other via the phosphorus atom P¹ or P²; and wherein the other pair R⁵ and R⁶ together or R¹ and R² together represent an organic bivalent radical linked to the phosphorus atom P² or P¹ via tertiary carbon atoms.

15. A catalyst composition comprising:
(a) a source of a metal of group VIII, and
(b) the bidentate diphosphine ligand of claim 14.

16. The catalyst composition of claim 15 wherein in the bidentate diphosphine ligand the dihedral angle between the plane occupied by the three atom sequence composed of C¹, C² and the atom directly bonded to C¹ in direction of P¹, and the plane occupied by the three atom sequence C¹, C² and the atom directly bonded to C² in direction of P², is in the range of from 0 to 120°.

17. A carbonylation product composition containing less than 1.5 ppmw of nitrogen-containing impurities and less than 1.5 ppmw of halogen-containing impurities; and wherein the product composition is derived from 1,3-butadiene and contains α-methyl glutaric acid and/or α-ethyl succinic acid; and wherein the product composition contains less than 1.5 ppmw of glutaric acid and/or succinic acid.

18. The process of claim 1 wherein the bond is restricted at a temperature in the range of 50° C. to 250° C.
